(19) United States
(12) Patent Application Publication
(10) Pub. No.: US 2024/0148514 A1
(43) Pub. Date: May 9, 2024

(54) SPINAL IMPLANT

(71) Applicant: Evolution Spine, Dallas, TX (US)

(72) Inventors: Benjamin Keller, Austin, TX (US); Todd Wallenstein, Ashburn, VA (US); Francia Sanchez, Knoxville, TN (US); James Mirda, Germantown, TN (US)

(21) Appl. No.: 18/499,751

(22) Filed: Nov. 1, 2023

Related U.S. Application Data
(60) Provisional application No. 63/422,044, filed on Nov. 3, 2022.

(51) Int. Cl. A61F 2/44; A61F 2/30

(52) U.S. Cl. CPC A61F 2/44; A61F 2/30767; A61F 2/3094; A61F 2002/3093; A61F 2002/30968; A61F 2002/30985

(57) ABSTRACT

A spinal implant comprising a major branched lattice formed in at least a portion of a middle portion of the spinal implant; and a minor branched lattice formed throughout the major branched lattice.

### CROSS-REFERENCE TO RELATED APPLICATIONS

This application claims priority to U.S. Provisional Application Ser. No. 63/422,044, filed Nov. 3, 2022, the entire contents of which are incorporated herein by reference.

### TECHNICAL FIELD OF THE INVENTION

The present invention relates in general to orthopedic implants and, more particularly, to spinal implants.

### STATEMENT OF FEDERALLY FUNDED RESEARCH

Not applicable.

### INCORPORATION-BY-REFERENCE OF MATERIALS FILED ON COMPACT DISK

Not applicable.

### BACKGROUND OF THE INVENTION

Without limiting the scope of the invention, its background is described in connection with a spinal implant as an example.

An intervertebral disc in human spine may be replaced by a spinal implant to promote fusion of the vertebrae adjacent to the disc.

U.S. Pat. No. 11,273,048, to Cain et al., is said to disclose an orthopedic implant which generally includes a frame structure and a porous structure, in which both the frame and porous structure at least partially define at least six surfaces which make a three-dimensional profile of the implant, and in which the porous structure is positioned at least partially within the three-dimensional profile.

U.S. Pat. App. Pub. No. 2022/0117753, by Rucker et al., is said to disclose a surgical implant device, including: a solid surface; and a lattice structure disposed adjacent to the solid surface, wherein the lattice structure includes a first plurality of struts that define a first plurality of voids adjacent to the solid surface and a second plurality of struts that define a second plurality of voids remote from the solid surface. Each of the first plurality of struts is said to have an average cross-sectional diameter that is smaller than an average cross-sectional diameter of each of the second plurality of struts. Each of the first plurality of voids is said to have an average internal diameter that is smaller than an average internal diameter of each of the second plurality of voids. The surgical implant device is also said to include a needle-populated porous surface disposed adjacent to the solid surface opposite the lattice structure.

### SUMMARY OF THE INVENTION

An embodiment of the present invention includes a spinal implant including a major branched lattice formed in at least a portion of a middle portion of the spinal implant; and; and a minor branched lattice formed throughout the major branched lattice. In one aspect, the spinal implant is flat or wedge-shaped. In another aspect, a front face of the spinal implant is closed. In another aspect, the spinal implant includes a stainless steel, a titanium alloy, an aluminum alloy, a chromium alloy, a metal alloy, CoCrMo, Hydroxyapatite, a polyether ether ketone (PEEK) material, a polyether ketone ketone (PEKK), a carbon fiber material, an ABS plastic, a polyurethane, a polyethylene, a photo-polymer, a resin, a fiber-encased resinous material, a latex, a synthetic rubber, a synthetic material, a polymer, or a natural material, or another biocompatible material. In another aspect, the spinal implant is made at least in part by rapid prototyping, 3D printing, stereolithography (STL), selective laser sintering (SLS), fused deposition modeling (FDM), direct metal laser sintering (DMLS), electron beam melting (EBM), multi-jet fusion (MJF), or an additive manufacturing machine. In another aspect, surfaces of the minor branched lattice are coated with a bone growth-promoting agent. In another aspect, the bone growth-promoting agent comprises Hydroxyapatite (HA). In another aspect, the each of the major branched lattice or the minor branched lattice, or both, are configured to reduce or prevent leakage of the bone growth-promoting agent from the spinal implant. In another aspect, the major branched lattice, the minor branched lattice, or both, are a Voronoi lattice. In another aspect, the major branched lattice, the minor branched lattice, or both, are a Gyroid lattice. In another aspect, the major branched lattice, the minor branched lattice, or both, are a diamond lattice. In another aspect, the major branched lattice, the minor branched lattice, or both, are a Schwarz lattice. In another aspect, the major branched lattice, the minor branched lattice, or both, are a Split P lattice. In another aspect, the major branched lattice, the minor branched lattice, or both, include two or more different types of lattices. In another aspect, there are no supporting structures surrounding the major, the minor, or both the major and minor lattices. In another aspect, a roughness is applied to surfaces of the minor branched lattice. In another aspect, the spinal implant further includes one or more openings in the spinal implant for fasteners to secure the spinal implant to one or more vertebrae. In another aspect, the spinal implant further includes one or more openings for fasteners comprising a locking tab to secure a fastener. In another aspect, the spinal implant further includes one or more instrument ports.

Another embodiment of the present invention includes a method of using a spinal implant including providing a patient in need of the spinal implant; providing the spinal implant including: a major branched lattice formed in at least a portion of a middle portion of the spinal implant; and a minor branched lattice formed throughout the major branched lattice; and implanting the spinal implant into a spine of the patient. In one aspect, the spinal implant is flat or wedge-shaped. In another aspect, a front face of the spinal implant is closed. In another aspect, spinal implant includes a stainless steel, a titanium alloy, an aluminum alloy, a chromium alloy, a metal alloy, CoCrMo, Hydroxyapatite, a polyether ether ketone (PEEK) material, a polyether ketone ketone (PEKK), a carbon fiber material, an ABS plastic, a polyurethane, a polyethylene, a photo-polymer, a resin, a fiber-encased resinous material, a latex, a synthetic rubber, a synthetic material, a polymer, or a natural material, or another biocompatible material. In another aspect, the spinal implant is made at least in part by rapid prototyping, 3D printing, stereolithography (STL), selective laser sintering (SLS), fused deposition modeling (FDM), direct metal laser sintering (DMLS), electron beam melting (EBM), multi-jet fusion (MJF), or an additive manufacturing machine. In another aspect, surfaces of the minor branched lattice are coated with a bone growth-promoting agent. In another aspect, the bone growth-promoting agent comprises Hydroxyapatite (HA). In another aspect, the major branched lattice, the minor branched lattice, or both are configured to reduce or prevent leakage of a bone growth-promoting agent from the spinal implant. In another aspect, the major branched lattice, the minor branched lattice, or both are a Voronoi lattice. In another aspect, the major branched lattice, the minor branched lattice, or both are a Gyroid lattice. In another aspect, the major branched lattice, the minor branched lattice, or both are a diamond lattice. In another aspect, the major branched lattice, the minor branched lattice, or both are a Schwarz lattice. In another aspect, the major branched lattice, the minor branched lattice, or both are a Split P lattice. In another aspect, the major branched lattice, the minor branched lattice, or both include two or more different types of lattices. In another aspect, a roughness is applied to surfaces of the minor branched lattice. In another aspect, the spinal implant further includes one or more openings for fasteners to secure the spinal implant to one or more vertebrae. In another aspect, each of the one or more openings for fasteners includes a locking tab to secure a fastener. In another aspect, the spinal implant further includes instrument ports.

Another embodiment of the present invention includes a method of making a spinal implant including forming a major branched lattice in at least a portion of a middle portion of the spinal implant; and forming a minor branched lattice throughout the major branched lattice. In one aspect, the method further includes coating surfaces of the minor branched lattice with a bone growth-promoting agent. In another aspect, a front face of the spinal implant is closed. In another aspect, the spinal implant includes a stainless steel, a titanium alloy, an aluminum alloy, a chromium alloy, a metal alloy, CoCrMo, Hydroxyapatite, a polyether ether ketone (PEEK) material, a polyether ketone (PEKK), a carbon fiber material, an ABS plastic, a polyurethane, a polyethylene, a photo-polymer, a resin, a fiber-encased resinous material, a latex, a synthetic rubber, a synthetic material, a polymer, or a natural material, or another biocompatible material. In another aspect, the spinal implant is made at least in part by rapid prototyping, 3D printing, stereolithography (STL), selective laser sintering (SLS), fused deposition modeling (FDM), direct metal laser sintering (DMLS), electron beam melting (EBM), multi-jet fusion (MJF), or an additive manufacturing machine.

### BRIEF DESCRIPTION OF THE DRAWINGS

For a more complete understanding of the features and advantages of the present invention, reference is now made to the detailed description of the invention along with the accompanying figures and in which:

FIG. 1A shows an isometric view of an embodiment of the spinal implant. FIG. 1B shows a front view of the embodiment.

FIG. 2 shows a top view of the spinal implant.

FIG. 3 shows a side view of the spinal implant.

FIG. 4 shows an isometric cross-sectional view of the spinal implant taken through section A of the top view of FIG. 2.

FIG. 5 shows a side cross-sectional view of the spinal implant taken through section A of the top view of FIG. 2.

FIG. 6 shows an isometric cross-sectional view of the spinal implant taken through section B of the side view of FIG. 3.

FIG. 7A shows top, isometric, front, and side views of an embodiment of the spinal implant without integrated fastener openings.

FIG. 7B shows a close-up top view of the spinal implant showing a roughness applied to the major branched lattice and the minor branched lattice.

FIG. 8A shows an isometric view of an alternative embodiment showing the major branched lattice, onto which a smaller gyroid lattice is generated, with no supporting structure.

FIG. 8B shows an isometric view of an alternative embodiment showing the major branched lattice, onto which a smaller diamond lattice is generated, with no supporting structure.

FIG. 8C shows an isometric view of an alternative embodiment showing the major branched lattice, onto which a smaller Schwartz lattice is generated, with no supporting structure.

FIG. 9A shows a closeup view of the fastener openings encompassed within the spinal implant showing multiple raised depressions onto which an instrument can mate.

FIG. 9B shows a closeup view of the fastener openings encompassed within the spinal implant showing a single raised depression onto which an instrument can mate.

FIG. 10 shows a flowchart for a method embodiment of the present invention.

FIG. 11 shows a flowchart for another method embodiment of the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The terminology used and specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

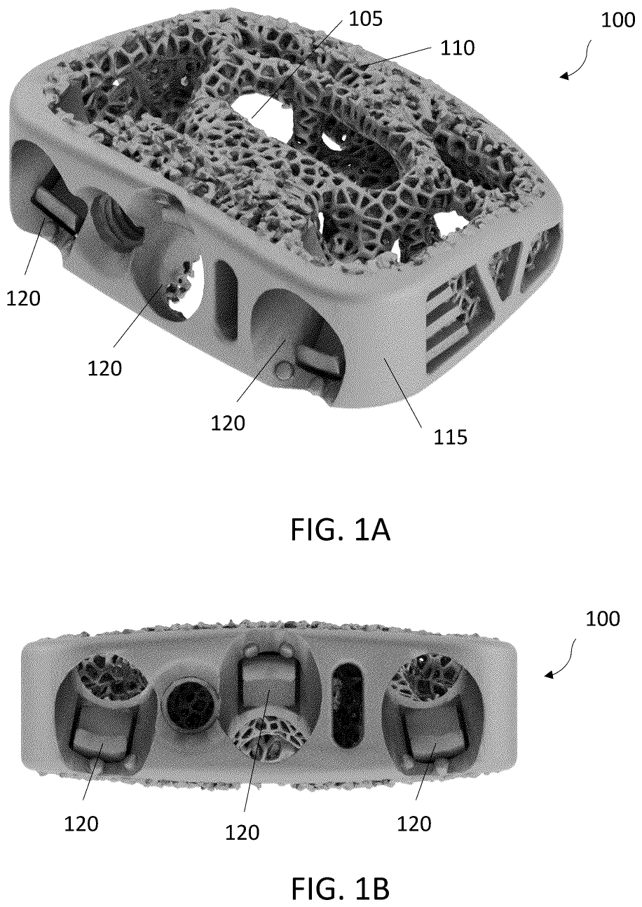

FIG. 1A shows an isometric view of an embodiment of the spinal implant 100. FIG. 1B shows a front view of the embodiment. As illustrated in FIG. 1A, the spinal implant includes a major branched lattice 105, throughout which a minor branched lattice 110 is formed. In the embodiment shown, the major branched lattice 105 is formed throughout a middle portion of the spinal implant 100. As shown, the major branched lattice 105 and the minor branched lattice 110 are both lattices of Voronoi form, but each lattice can take other forms as discussed herein. The major branched lattice 105 and the minor branched lattice 110 may each vary in size and density. Within each lattice, the number of lumens, the number of branches, the density of the struts, and the thickness of the struts may vary. The spinal implant 100 may include a perimeter band 115. The spinal implant may also include fasteners openings 120 for use in securing the spinal implant to one or more vertebrae using fasteners (not shown). FIG. 1B shows the fastener openings 120.

One advantage of utilizing multiple lattice structures is to encourage and maximize the bone growth through the device. The major branched lattice 105 allows for easier loading of bone graft within the cage. The major branched lattice 105 creates an open architecture that also promotes mechanical load sharing of the spinal implant 100 to the surrounding vertebral bodies. While prior art exists that utilize some form of internal architecture to allow for load sharing, the overall goal of these devices is to promote new bone formation. To promote new bone formation, the present invention adds a minor branched lattice 110 formed throughout the major branched lattice 105. This minor branched lattice 110 allows for angiogenesis (new vessel formation) to occur onto and into the major branched lattice 105. Prior art does not account for this. The minor branched lattice 110 allows for an optimized pore size for angiogenesis of between 300 to 600 μm, which one familiar in the art of bone formation would know, based on peer reviewed literature, is the ideal range for angiogenesis. In addition, the open architecture of the minor branched lattice 110 also allows for the construction of a highly porous device in the >70% porosity range. The higher the porosity, the more volume of space is available for bone to form within the spinal implant 100. Large lattice structures on the prior art are solid which thereby reduce the porosity and pore size. As the porosity of the structure increases, the stiffness of the spinal implant 100 can also be modified to match the stiffness of the vertebral endplates more closely. Someone who is familiar with state of art knows that a highly stiff device has a higher probability of subsiding into the vertebral body endplates. Prior art devices can only modify the stiffness of the device by changing the size, shape, and density of the larger struts. Changing the strut size may compromise the bone packing ability, reduce the angiogenesis potential, or result in a suboptimal stiffness. Adding the minor branched lattice structure 110 reduces or eliminates those issues. In addition to the minor branched lattice structure 110, a roughness (not shown) may be added to create a more wettable surface that allows for protein adsorption and blood clot formation. Based on peer reviewed literature this occurs at around a roughness of 1-4 μm. A roughness can be applied to surfaces of the minor branched lattice 110 to create this hydrophilic surface area.

The spinal implant 100 may be flat or wedge-shaped. In some embodiments of the spinal implant 100, the front face (as oriented when implanted in a patient) of the perimeter band 115 is closed to prevent leakage of a biological growth-promoting agent. The spinal implant 100 is made of a biocompatible material, such as a stainless steel, a titanium alloy, an aluminum alloy, a chromium alloy, a metal alloy, CoCrMo, Hydroxyapatite, a polyether ether ketone (PEEK) material, a polyether ketone ketone (PEKK), a carbon fiber material, an ABS plastic, a polyurethane, a polyethylene, a photo-polymer, a resin, a fiber-encased resinous material, a latex, a synthetic rubber, a synthetic material, a polymer, or a natural material, or another biocompatible material. The spinal implant 100 may be produced at least in part by rapid prototyping, 3D printing, stereolithography (STL), selective laser sintering (SLS), fused deposition modeling (FDM), direct metal laser sintering (DMLS), electron beam melting (EBM), multi-jet fusion (MJF), or an additive manufacturing machine.

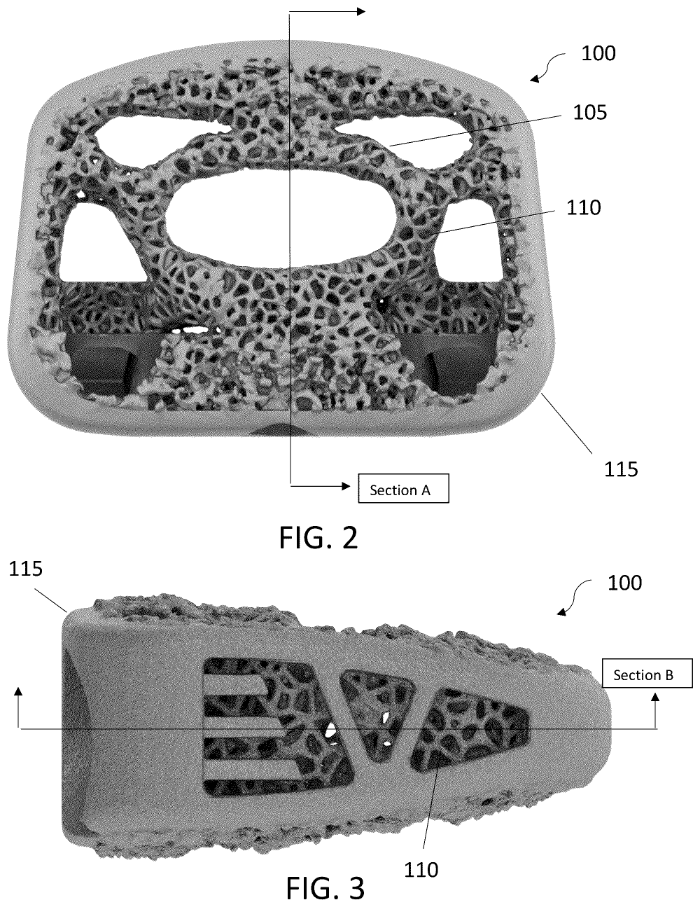

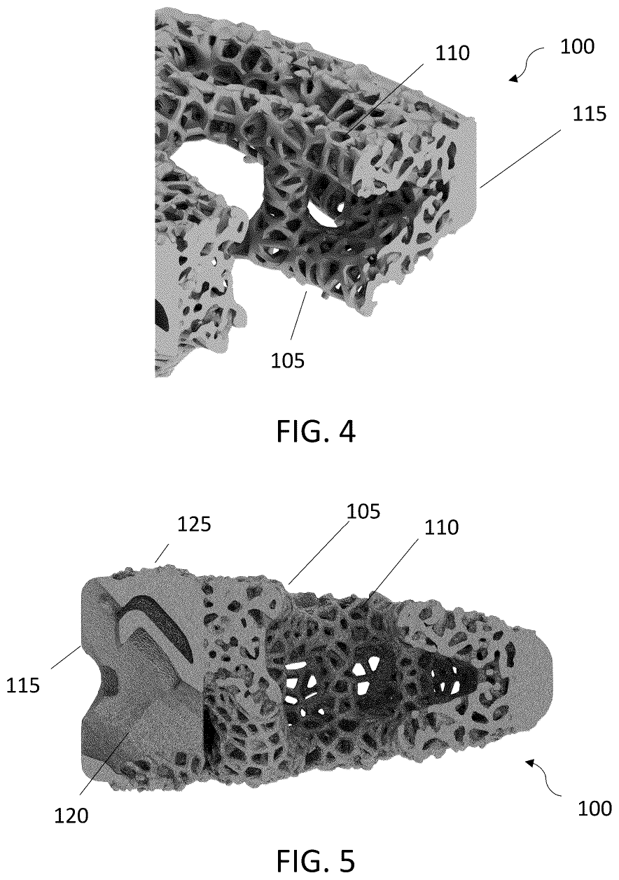

FIG. 2 shows a top view of the spinal implant1 100, with the major branched lattice 105, the minor branches lattice 110, the perimeter band 115, and a section A through the spinal implant 100. FIG. 3 shows a side view of the spinal implant 100, with the minor branched lattice 110, the perimeter band 115, and a section B through the spinal implant 100. FIG. 4 shows an isometric cross-sectional view of the spinal implant 100 taken at section A of the top view of FIG. 2, with the major branched lattice 105, the minor branched lattice 110, and the perimeter band 115.

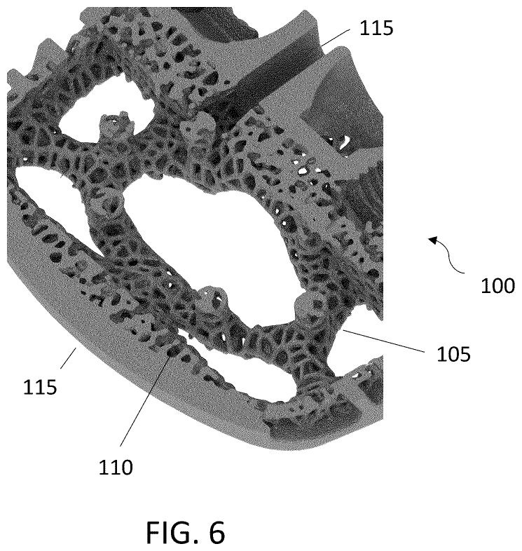

FIG. 5 shows a side cross-sectional view of the spinal implant 100 taken at section A of the top view of FIG. 2, with the major branched lattice 105, the minor branched lattice 110, the perimeter band 115, a fastener opening 120, and the roughness 125. The roughness 125 may vary in amplitude, height, width, and density of roughness. FIG. 6 shows an isometric cross-sectional view of the spinal implant taken through section B of the side view of FIG. 3, with the major branched lattice 105, the minor branched lattice 110, and the perimeter band 115. In the embodiment shown in FIGS. 1-6, the major branched lattice 105 is formed throughout the middle portion of the spinal implant 100. In other embodiments, the major branched lattice 105 is formed in only a portion of the middle portion of the spinal implant 100, or the major branched lattice 105 is formed to extend to the exterior of the spinal implant 100 without a perimeter band 115.

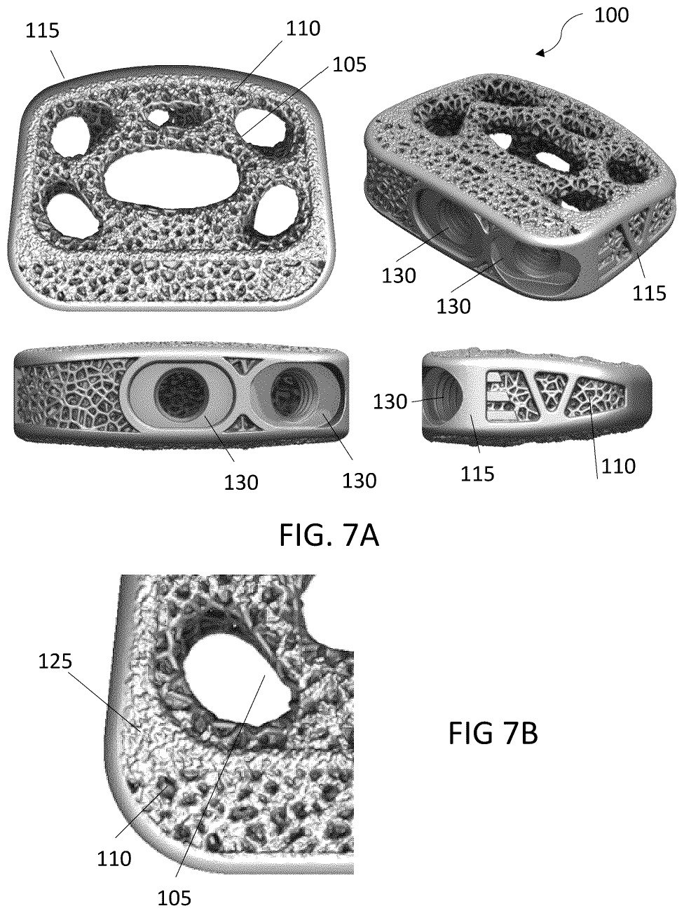

FIG. 7A shows top (upper left), isometric (top right), front (lower left), and side (lower right) views of an embodiment of the spinal implant 100 without the fastener openings 120, and with the major branched lattice 105, the minor branched lattice 110, the perimeter band 115 and instrument ports 130. FIG. 7B shows a close-up of the top view of the spinal implant 100 showing the roughness 125.

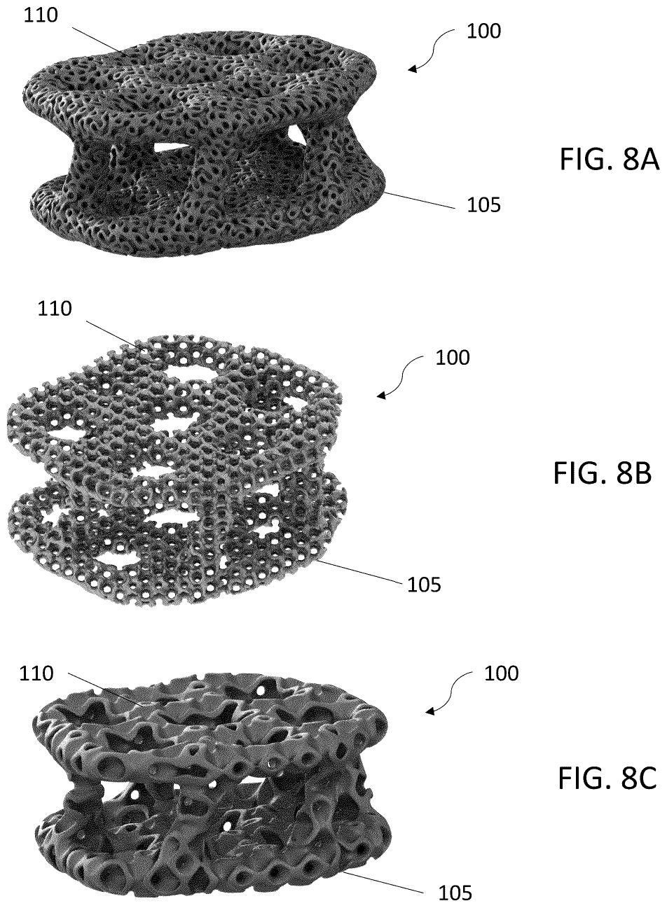

FIG. 8A shows an isometric view of an alternative embodiment of the spinal implant 100 showing the major branched lattice 105, onto which a minor branched lattice 110 of gyroid form is generated, with no supporting structure. FIG. 8B shows an isometric view of an alternative embodiment of the spinal implant 100 showing the major branched lattice 105, onto which a minor branched lattice 110 of diamond form is generated, with no supporting structure. FIG. 8C shows an isometric view of an alternative embodiment of the spinal implant 100 showing the major branched lattice 105, onto which a minor branched lattice 110 of Schwartz form is generated, with no supporting structure. Either or both of the major branched lattice 105 or the minor branched lattice 110 can also take other forms, such as a split-P form (not shown).

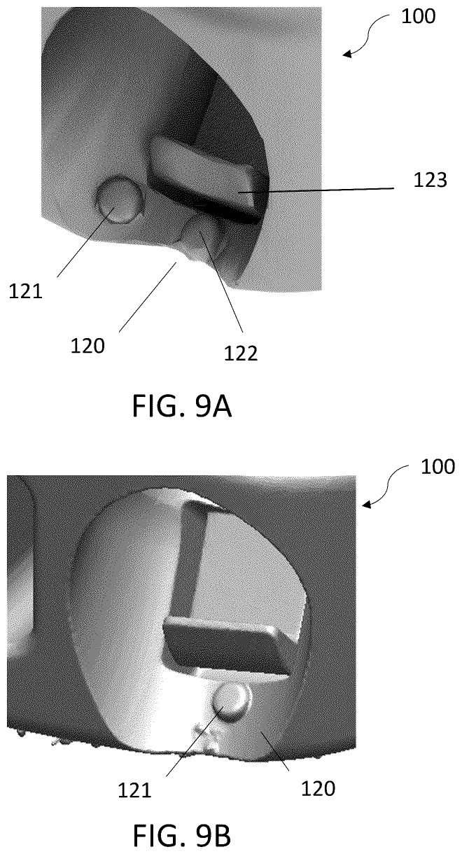

FIG. 9A shows a closeup view of the fastener openings 120 showing exemplary instrument ports 121, 122 in the form of raised depressions, onto which an instrument (not shown) can mate. FIG. 9A also shows a locking tab 123 to secure a fastener. FIG. 9B shows a closeup view of a fastener opening 120 with a single instrument port 121 in the form of a raised depression onto which an instrument (not shown) can mate. Each instrument port may be circular or spherical in shape, or may take a variety of other shapes.

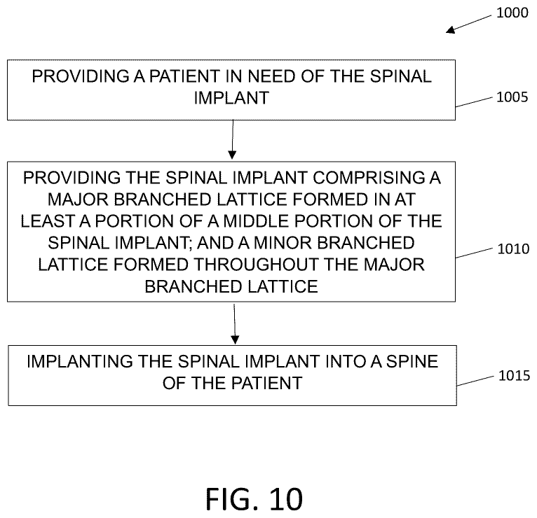

FIG. 10 shows a flowchart for a method embodiment of the present invention. Method 1000, a method of using a spinal implant, includes block 1005, providing a patient in need of the spinal implant. Method 1000 further includes block 1010, providing the spinal implant including a major branched lattice formed in at least a portion of a middle portion of the spinal implant; and a minor branched lattice formed throughout the major branched lattice. In addition, Method 1000 includes block 1015, implanting the spinal implant into a spine of the patient.

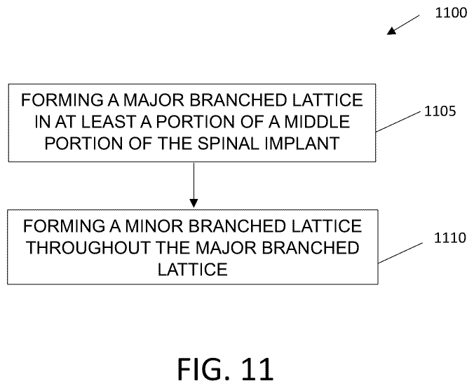

FIG. 11 shows a flowchart for another method embodiment of the present invention. Method 1100, a method of making a spinal implant, includes block 1105, forming a major branched lattice in at least a portion of a middle portion of the spinal implant; and block 1110, forming a minor branched lattice throughout the major branched lattice.

In one embodiment, the present invention also includes a spinal implant comprising: a perimeter band comprising an upper ring, a lower ring, and a major branched lattice structure of perimeter cells of random cell size disposed between the upper ring and the lower ring; and a major branched lattice formed in at least a portion of a middle portion of the spinal implant; and a minor branched lattice formed throughout the major branched lattice; and an interior portion disposed within the major branched lattice, wherein the interior portion comprises a minor branched lattice structure of interior cells, wherein the interior minor branched lattice structure and one or more internal support members are connected to or integral with the upper ring or the lower ring to support the minor branched lattice structure.

An embodiment of the present invention comprises, consists essentially of, or consists of a spinal implant including a major branched lattice formed in at least a portion of a middle portion of the spinal implant; and; and a minor branched lattice formed throughout the major branched lattice. In one aspect, the spinal implant is flat or wedge-shaped. In another aspect, a front face of the spinal implant is closed. In another aspect, the spinal implant includes a stainless steel, a titanium alloy, an aluminum alloy, a chromium alloy, a metal alloy, CoCrMo, Hydroxyapatite, a polyether ether ketone (PEEK) material, a polyether ketone (PEKK), a carbon fiber material, an ABS plastic, a polyurethane, a polyethylene, a photo-polymer, a resin, a fiber-encased resinous material, a latex, a synthetic rubber, a synthetic material, a polymer, or a natural material, or another biocompatible material. In another aspect, the spinal implant is made at least in part by rapid prototyping, 3D printing, stereolithography (STL), selective laser sintering (SLS), fused deposition modeling (FDM), direct metal laser sintering (DMLS), electron beam melting (EBM), multi-jet fusion (MJF), or an additive manufacturing machine. In another aspect, surfaces of the minor branched lattice are coated with a bone growth-promoting agent. In another aspect, the bone growth-promoting agent comprises Hydroxyapatite (HA). In another aspect, the each of the major branched lattice or the minor branched lattice, or both, are configured to reduce or prevent leakage of the bone growth-promoting agent from the spinal implant. In another aspect, the major branched lattice, the minor branched lattice, or both, are a Voronoi lattice. In another aspect, the major branched lattice, the minor branched lattice, or both, are a Gyroid lattice. In another aspect, the major branched lattice, the minor branched lattice, or both, are a diamond lattice. In another aspect, the major branched lattice, the minor branched lattice, or both, are a Schwarz lattice. In another aspect, the major branched lattice, the minor branched lattice, or both, are a Split P lattice. In another aspect, the major branched lattice, the minor branched lattice, or both, include two or more different types of lattices. In another aspect, there are no supporting structures surrounding the major, the minor, or both the major and minor lattices. In another aspect, a roughness is applied to surfaces of the minor branched lattice. In another aspect, the spinal implant further includes one or more openings in the spinal implant for fasteners to secure the spinal implant to one or more vertebrae. In another aspect, the spinal implant further includes one or more openings for fasteners comprising a locking tab to secure a fastener. In another aspect, the spinal implant further includes one or more instrument ports.

Another embodiment of the present invention comprises, consists essentially of, or consists of a method of using a spinal implant including providing a patient in need of the spinal implant; providing the spinal implant including: a major branched lattice formed in at least a portion of a middle portion of the spinal implant; and a minor branched lattice formed throughout the major branched lattice; and implanting the spinal implant into a spine of the patient. In one aspect, the spinal implant is flat or wedge-shaped. In another aspect, a front face of the spinal implant is closed. In another aspect, spinal implant includes a stainless steel, a titanium alloy, an aluminum alloy, a chromium alloy, a metal alloy, CoCrMo, Hydroxyapatite, a polyether ether ketone (PEEK) material, a polyether ketone ketone (PEKK), a carbon fiber material, an ABS plastic, a polyurethane, a polyethylene, a photo-polymer, a resin, a fiber-encased resinous material, a latex, a synthetic rubber, a synthetic material, a polymer, or a natural material, or another biocompatible material. In another aspect, the spinal implant is made at least in part by rapid prototyping, 3D printing, stereolithography (STL), selective laser sintering (SLS), fused deposition modeling (FDM), direct metal laser sintering (DMLS), electron beam melting (EBM), multi-jet fusion (MJF), or an additive manufacturing machine. In another aspect, surfaces of the minor branched lattice are coated with a bone growth-promoting agent. In another aspect, the bone growth-promoting agent comprises Hydroxyapatite (HA). In another aspect, the major branched lattice, the minor branched lattice, or both are configured to reduce or prevent leakage of a bone growth-promoting agent from the spinal implant. In another aspect, the major branched lattice, the minor branched lattice, or both are a Voronoi lattice. In another aspect, the major branched lattice, the minor branched lattice, or both are a Gyroid lattice. In another aspect, the major branched lattice, the minor branched lattice, or both are a diamond lattice. In another aspect, the major branched lattice, the minor branched lattice, or both are a Schwarz lattice. In another aspect, the major branched lattice, the minor branched lattice, or both are a Split P lattice. In another aspect, the major branched lattice, the minor branched lattice, or both include two or more different types of lattices. In another aspect, a roughness is applied to surfaces of the minor branched lattice. In another aspect, the spinal implant further includes one or more openings for fasteners to secure the spinal implant to one or more vertebrae. In another aspect, each of the one or more openings for fasteners includes a locking tab to secure a fastener. In another aspect, the spinal implant further includes instrument ports.

Another embodiment of the present invention comprises, consists essentially of, or consists of a method of making a spinal implant including forming a major branched lattice in at least a portion of a middle portion of the spinal implant; and forming a minor branched lattice throughout the major branched lattice. In one aspect, the method further includes coating surfaces of the minor branched lattice with a bone growth-promoting agent. In another aspect, a front face of the spinal implant is closed. In another aspect, the spinal implant includes a stainless steel, a titanium alloy, an aluminum alloy, a chromium alloy, a metal alloy, CoCrMo, Hydroxyapatite, a polyether ether ketone (PEEK) material, a polyether ketone ketone (PEKK), a carbon fiber material, an ABS plastic, a polyurethane, a polyethylene, a photo-polymer, a resin, a fiber-encased resinous material, a latex, a synthetic rubber, a synthetic material, a polymer, or a natural material, or another biocompatible material. In another aspect, the spinal implant is made at least in part by rapid prototyping, 3D printing, stereolithography (STL), selective laser sintering (SLS), fused deposition modeling (FDM), direct metal laser sintering (DMLS), electron beam melting (EBM), multi-jet fusion (MJF), or an additive manufacturing machine.

It is contemplated that any embodiment discussed in this specification can be implemented with respect to any method, kit, apparatus or system of the invention, and vice versa.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A spinal implant comprising:
a major branched lattice formed in at least a portion of a middle portion of the spinal implant; and
a minor branched lattice formed throughout the major branched lattice.

2. The spinal implant of claim 1, wherein the spinal implant is flat or wedge-shaped.

3. The spinal implant of claim 1, wherein a front face of the spinal implant is closed.

4. The spinal implant of claim 1, wherein the spinal implant comprises a stainless steel, a titanium alloy, an aluminum alloy, a chromium alloy, a metal alloy, CoCrMo, Hydroxyapatite, a polyether ether ketone (PEEK) material, a polyether ketone ketone (PEKK), a carbon fiber material, an ABS plastic, a polyurethane, a polyethylene, a photo-polymer, a resin, a fiber-encased resinous material, a latex, a synthetic rubber, a synthetic material, a polymer, or a natural material, or another biocompatible material.

5. The spinal implant of claim 1, wherein the spinal implant is made at least in part by rapid prototyping, 3D printing, stereolithography (STL), selective laser sintering (SLS), fused deposition modeling (FDM), direct metal laser sintering (DMLS), electron beam melting (EBM), multi-jet fusion (MJF), or an additive manufacturing machine.

6. The spinal implant of claim 1, wherein surfaces of the minor branched lattice are coated with a bone growth-promoting agent.

7. The spinal implant of claim 6, wherein the bone growth-promoting agent comprises Hydroxyapatite (HA).

8. The spinal implant of claim 6, wherein the each of the major branched lattice or the minor branched lattice, or both, are at least one of:
configured to reduce or prevent leakage of the bone growth-promoting agent from the spinal implant;
a Voronoi lattice;
a Gyroid lattice;
a diamond lattice;
a Schwarz lattice; or
a Split P lattice; or
comprises two or more different types of lattices.

9. The spinal implant of claim 8, wherein there are no supporting structures surrounding the major, the minor, or both the major and minor lattices.

10. The spinal implant of claim 1, wherein a roughness is applied to surfaces of the minor branched lattice.

11. The spinal implant of claim 1, further comprising at least one of:
one or more openings in the spinal implant for fasteners to secure the spinal implant to one or more vertebrae;
one or more openings for fasteners comprising a locking tab to secure a fastener; or one or more instrument ports.

12. A method of using a spinal implant comprising:
providing a patient in need of the spinal implant;
providing the spinal implant comprising:
a major branched lattice formed in at least a portion of a middle portion of the spinal implant; and
a minor branched lattice formed throughout the major branched lattice; and
implanting the spinal implant into a spine of the patient.

13. The method of claim 12, wherein the spinal implant is flat or wedge-shaped.

14. The method of claim 12, wherein a front face of the spinal implant is closed.

15. The method of claim 12, the spinal implant comprises a stainless steel, a titanium alloy, an aluminum alloy, a chromium alloy, a metal alloy, CoCrMo, Hydroxyapatite, a polyether ether ketone (PEEK) material, a polyether ketone ketone (PEKK), a carbon fiber material, an ABS plastic, a polyurethane, a polyethylene, a photo-polymer, a resin, a fiber-encased resinous material, a latex, a synthetic rubber, a synthetic material, a polymer, or a natural material, or another biocompatible material.

16. The method of claim 12, wherein the spinal implant is made at least in part by rapid prototyping, 3D printing, stereolithography (STL), selective laser sintering (SLS), fused deposition modeling (FDM), direct metal laser sintering (DMLS), electron beam melting (EBM), multi-jet fusion (MJF), or an additive manufacturing machine.

17. The method of claim 12, wherein surfaces of the minor branched lattice are coated with a bone growth-promoting agent.

18. The method of claim 17, wherein the bone growth-promoting agent comprises Hydroxyapatite (HA).

19. The method of claim 12, wherein each of the major branched lattice or the minor branched lattice, or both, are at least one of:
configured to reduce or prevent leakage of a bone growth-promoting agent from the spinal implant;
a Voronoi lattice;
a Gyroid lattice;
a diamond lattice;
a Schwarz lattice; or
a Split P lattice; or
comprises two or more different types of lattices.

20. The method of claim 12, wherein at least one of:
a roughness is applied to surfaces of the minor branched lattice;
the spinal implant comprises one or more openings for fasteners to secure the spinal implant to one or more vertebrae; or
each of the one or more openings for fasteners comprises a locking tab to secure a fastener.

21. The method of claim 12, further comprising instrument ports.

22. A method of making a spinal implant comprising:
forming a major branched lattice in at least a portion of a middle portion of the spinal implant; and
forming a minor branched lattice throughout the major branched lattice.

23. The method of claim 22, further comprising coating surfaces of the minor branched lattice with a bone growth-promoting agent.

24. The method of claim 22, wherein a front face of the spinal implant is closed.

25. The method of claim 22, wherein the spinal implant comprises a stainless steel, a titanium alloy, an aluminum alloy, a chromium alloy, a metal alloy, CoCrMo, Hydroxyapatite, a polyether ether ketone (PEEK) material, a polyether ketone ketone (PEKK), a carbon fiber material, an ABS plastic, a polyurethane, a polyethylene, a photo-polymer, a resin, a fiber-encased resinous material, a latex, a synthetic rubber, a synthetic material, a polymer, or a natural material, or another biocompatible material.

26. The method of claim 22, wherein the spinal implant is made at least in part by rapid prototyping, 3D printing, stereolithography (STL), selective laser sintering (SLS), fused deposition modeling (FDM), direct metal laser sintering (DMLS), electron beam melting (EBM), multi-jet fusion (MJF), or an additive manufacturing machine.
